(19) United States
(12) Patent Application Publication
(10) Pub. No.: US 2010/0161015 A1
(43) Pub. Date: Jun. 24, 2010

(54) K9 Comfort Therm-a-Pack

(76) Inventors: Kimberly D. Wilson, Portland, OR (US); Angela M. McHarness, Portland, OR (US)

Correspondence Address: Kimberly D. Wilson, 143 SE 109th Ave., Portland, OR, 97216, US

(21) Appl. No.: 12/317,174

(22) Filed: Dec. 22, 2008

(51) Int. Cl. A61F 7/08; A61F 7/10

(52) U.S. Cl. 607/112

(57) ABSTRACT

K9 Therm-a-pack is designed to apply ice or heat packs to a dog's hip or lower back area with out the owner having to bold it in place. Comprised of water proof or resistant material to keep the dogs skin dry, two pockets hold the ice/heat pack in place while three adjustable straps secure the pack in place on the dog. Two straps go around the dog's hind legs and one around the waist attaching with a plastic clasp that allows the straps to be adjusted in length.

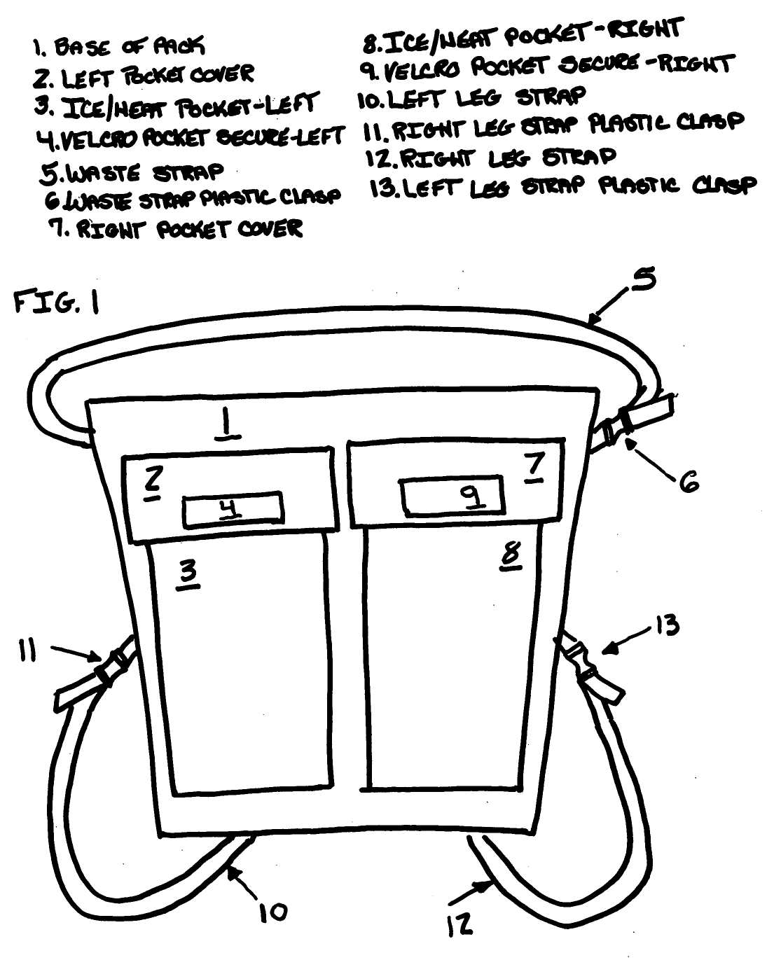

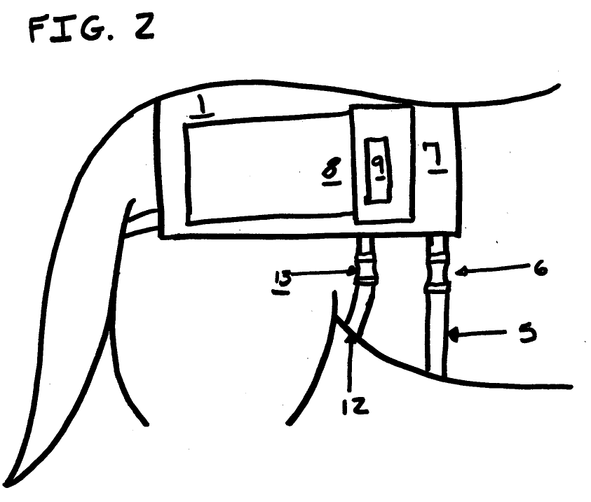

### BACKGROUND

The idea for the K9 Comfort Therm-a-pack originated while curing for our senior German Shepard mix that was suffering hip dysplasia. We tried multiple methods to apply heat and ice to his hind end with out having to sit and hold the pack on ourselves. My most successful method was putting the ice/heat pack on the dog's hips and wrapping an elastic back brace, around his waist to hold it in place. The ice pack stayed in place for twenty minutes with out me holding it. That got me started on modifying it to something more comfortable for the dog and easier for the owner to put on.

### SUMMARY

The K9 Comfort Therm-a-pack is designed to be worn by a dog with pockets over the hip and lower back area that heat or ice packs can be placed inside. The pockets hold the ice/heat packs in place while the adjustable straps around the legs and waist, hold the Therm-a-pack comfortably in place on the dog.

### DRAWINGS

FIG. one shows the top view of the K9 Comfort Therm-a-pack displaying both pockets for inserting the ice/heat packs as well as the adjustable waste and leg snaps that hold the pack in place.

FIG. two shows a side view of the pack as it would look on the dog. This right sided view shows the right side pocket resting over the dog's right hip and lower back area. The leg strap goes around the right leg attaching just in front of the right hind leg. The waist strap goes directly under the belly of the dog and attaches on the side near the waist of the dog, with a plastic clip. Both the waist and leg straps are adjustable in length at the plastic clip.

### DESCRIPTION

The Therm-a-pack is made of thin, water proof or water resistant material to keep moisture off the dog's fur and skin. The shape is to fit over the dog's hind end, from near the bottom of the rib cage to the base of the dog's tail and held in place by three adjustable nylon straps. Two straps run from the back corners of the garment (both sides of the dog's tail) on the inside of the dog's legs and attach approximately midway through the garment, in front of the dogs hind legs. The straps attach with a plastic clasp and are adjustable in length to accommodate a variety of different breeds and sizes. The third adjustable strap runs directly under the belly of the dog and also attaches with a plastic clasp. With the pack properly in place, the clasps for the straps lay on the side of the dog for easy access. On the top of the pack, the part resting on the dog's hind end, are two pockets placed to hold an ice or heat pack over the dogs hips. Once the ice/heat pack is inserted, the pocket covers fold down and close with a piece of Velcro to hold the ice/heat pack in place.

1. K9 Therm-a-pack is a unique design specifically for the use of applying heat or ice packs to your dog. Intended to be warn by the dog, it is comfortable, adjustable and does not require the owner or caregiver to hold the ice or heat pack in place.
